(19) United States
(12) Patent Application Publication
(10) Pub. No.: US 2011/0257487 A1
(43) Pub. Date: Oct. 20, 2011

(54) Lateral and Anterior Lateral Retractor System

(76) Inventors: John Thalgott, Las Vegas, NV (US); Christopher T. Martin, Empire, MI (US); Steve Nowak, Traverse City, MI (US); Josh Delickta, Elk Rapids, MI (US); Daniel K. Farley, Traverse City, MI (US); Stephanie Zalucha, Williamsburg, MI (US)

(21) Appl. No.: 13/087,866

(22) Filed: Apr. 15, 2011

Related U.S. Application Data
(60) Provisional application No. 61/324,501, filed on Apr. 15, 2010.

(51) Int. Cl. A61B 1/32

(52) U.S. Cl. 600/208

(57) ABSTRACT

A novel surgical lateral retraction system is provided, including a delivery device and retractor blades. The delivery device includes a shaft having distal and proximal portions and a plurality of engaging features extending along the distal portion of the shaft. The engaging features are sized and configured to slidingly engage the retractor blades, wherein each retractor blade may be attached to the shaft independently by sliding onto one of the engaging features. The shaft may include first and second curved surfaces on either side of at least one of the engaging features, wherein the first and second curved surfaces are sized and configured to correspond to retractor curved surfaces located on either side of a central channel of a retractor blade.

### RELATED APPLICATIONS

The present application claims priority to U.S. Provisional Patent Application 61/324,501, Attorney Docket No. 20931US01, entitled, “Lateral and Anterior Lateral Retractor System,” filed Apr. 15, 2010, the entire content of which is incorporated herein by reference.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The disclosed inventions relate to lateral and anterior lateral retractors and systems. In particular, the inventions relate to lateral and anterior lateral retractors and systems useful for minimally invasive surgery.

In the past, surgery typically required large incisions to provide access for visualization and instrument placement and manipulation relative to the surgical site. These large incisions could result in significant blood loss, damage to muscle tissue, increased healing times accompanied by prolonged pain, and significant scarring. Currently, many surgeries are conducted using minimally invasive techniques. These techniques minimize patient trauma by creating a relatively small incision, followed by the introduction of dilators to increase the effective size of the incision. Following dilation, surgery is performed through a surgical port inserted into the dilated incision. Instead of cutting through the muscle surrounding the surgical site, dilation effectively splits the muscle. Splitting, rather than cutting the muscle causes less damage to the muscle and leads to faster recovery times and reduced patient discomfort.

Dilators develop a channel from the subcutaneous layer of a patient to the site of operation. In certain procedures, a small incision, paralleling any underlying muscle, is made slightly longer than ½ the circumference of the largest dilator, or if used, port. A solid, pointed rod, variously described as a first dilator or guidewire, is then inserted into the incision to penetrate the underlying structures and reach the surgical site. It is best if the rod can be positioned against a bony surface as application of the dilators will attempt to push this rod forward. X-rays may be taken before and/or after insertion of the rod to confirm placement at the desired surgical site.

Increasingly larger diameter dilators can then be sequentially placed over each other to enlarge the channel. The larger diameters of the sequential dilators help to dilate the path of exposure while the series of tubes lessens the forces needed to create the path. The pointed tip of the dilators eases insertion and helps to widen the base of the channel when the dilator is orbited around a central axis formed through the center of the dilator along its length at the level of the skin.

In lieu of dilation, mechanical retractors can be used. Mechanical surgical retractors are hand-held or table-mounted metal retractor blades that are inserted into the incision, and thereafter retracted and held or locked in place to increase the effective opening of the incision. A drawback of using certain known retractors is that, in comparison to dilators, a relatively large incision must be made to provide for placement of the retractor blades.

Conventional dilators and surgical ports, however, are not suitable for all surgical applications. For example, conventional dilators are unable to completely dilate muscle away from the lamina of the spine due to the tortuous geometry of the lamina. Thus, muscle located between the dilator and the lamina must typically be cut away to access the lamina when using conventional dilators.

Due to the geometry of the spine, many spinal surgical procedures require a long, narrow opening. Thus, another drawback of ports is that a circular opening may not be practical for certain spinal surgeries because of the limited access it offers to the spine given the size of the dilated opening. The use of dilators and surgical ports may therefore be limited to procedures involving very precise access to the spine, such as for single level discectomy.

Mechanical retractors, on the other hand, may provide a long, narrow opening. As discussed above, however, mechanical retractors may require a relatively large initial incision that involves cutting, rather than splitting of muscle.

### BRIEF SUMMARY OF THE INVENTION

In certain embodiments of the present invention, a novel surgical lateral retraction system is provided. In certain embodiments, the lateral retraction system includes a plurality of retractor blades. The retractor blades include a distal tip shaped to abut a portion of a patient's spine. For example, the distal tip may include a concave, radiused surface sized to abut a portion of a patient's spine. A rearward surface of a distal portion of the blade includes a lip configured to hold back vessels.

Embodiments of the present invention include a delivery system for the plurality of retractor blades. Retractor blades are attachable to and removable from the delivery system. For example, the delivery system may deliver three retractor blades. The delivery system of certain embodiments includes a shaft to which the retractor blades are attached lengthwise, a distal portion adapted to accept a feature of a distal portion of the retractor blades, and a retention feature adapted to accept a portion of the retractor blades to secure the blades to the delivery system. The delivery system also includes a releasing mechanism to free the blades from the delivery system.

An embodiment of the present invention provides, for example, a method for providing access to a surgical site of interest. First, an incision is made. Then, a delivery system, with lateral retractor blades removably attached, is inserted into the incision. When the delivery system is fully inserted into the incision, a first retractor blade is removed from the delivery system, positioned in a desired orientation, and secured in place. Additional blades are subsequently removed from the delivery system and positioned as desired. After a desired number of blades have been positioned and secured, the delivery system is removed from the incision.

Embodiments of the present invention include a delivery device including a shaft having distal and proximal portions and a plurality of engaging features extending along the distal portion of the shaft. The engaging features are sized and configured to slidingly engage the retractor blades, wherein each retractor blade may be attached to the shaft independently by sliding onto one of the engaging features. In certain embodiments, the shaft includes first and second curved surfaces on either side of at least one of the engaging features, wherein the first and second curved surfaces are sized and configured to correspond to retractor curved surfaces located on either side of a central channel of a retractor blade.

An embodiment of the present invention provides a method for providing access to a surgical site of interest. The method includes making an incision. The method also includes inserting, into the incision, a shaft including engaging features for slidingly engaging retractor blades. Next, the method includes inserting, into the incision, a first retractor blade by sliding the first retractor blade along one of the engaging features, and inserting a second retractor blade by sliding the second retractor blade along a different one of the engaging features than the engaging feature along which the first retractor blade was inserted. Next, the method includes removing the shaft while leaving the first and second retractor blades in the incision, and then positioning the retractor blades as desired.

### BRIEF DESCRIPTION OF SEVERAL VIEWS OF THE DRAWINGS

FIG. 1 illustrates a side view of a retractor blade that may be used, for example, in an anterior lateral procedure, formed in accordance with an embodiment of the present invention.

FIG. 2 illustrates a rear view of the retractor blade of FIG. 1.

FIG. 3 illustrates an end view of the retractor blade of FIG. 1.

FIG. 4 illustrates a side view of a delivery system for retractor blades formed in accordance with an embodiment of the present invention.

FIG. 5 illustrates a sectional view of the delivery system of FIG. 4.

FIG. 6 illustrates a sectional view of the delivery system of FIG. 4.

FIG. 7 illustrates an end view of an anti-rotation collar formed in accordance with an embodiment of the present invention.

FIG. 8 illustrates an embodiment of a delivery system with retractor blades attached formed in accordance with an embodiment of the present invention.

FIG. 9 illustrates an embodiment of a retractor blade that may be used, for example, in an anterior lateral procedure, formed in accordance with an embodiment of the present invention in place after being positioned during a spinal procedure.

FIG. 10 illustrates a side view of a retractor blade that may be used, for example, in an anterior lateral procedure, formed in accordance with an embodiment of the present invention.

FIG. 11 illustrates a side sectional view of a retractor system adapted for delivery of different lengths of retractor blades formed in accordance with an embodiment of the present invention.

FIG. 12 provides an enlarged view of a portion of the sectional view of FIG. 11.

FIG. 13 illustrates a side view of a shuttle ball retainer formed in accordance with an embodiment of the present invention.

FIG. 14 illustrates a perspective view of a shuttle insertion head formed in accordance with an embodiment of the present invention.

FIG. 15 illustrates a side view of a grooved shaft formed in accordance with an embodiment of the present invention.

FIG. 16 illustrates a side sectional view of a shuttle ball lifter formed in accordance with an embodiment of the present invention.

FIG. 17 illustrates a sectional a retractor delivery system formed in accordance with another embodiment of the present invention.

FIG. 18 illustrates a perspective view of a retractor system that may be used, for example, to provide a lateral approach to the spine, formed in accordance with another embodiment of the present invention.

FIG. 19 illustrates a side view of the retractor system of FIG. 18.

FIG. 20 illustrates a side view of the retractor system of FIG. 18 with the retractor blades removed from the shuttle.

FIG. 21 illustrates an enlarged side view of the retractor system of FIG. 18 with the retractor blades removed from the shuttle.

FIG. 22 illustrates an enlarged perspective view of the retractor system of FIG. 18 with the retractor blades removed from the shuttle.

FIG. 23 illustrates a perspective view of a retractor delivery system formed in accordance with another embodiment of the present invention.

FIG. 24 illustrates a sectional view of the retractor delivery system of FIG. 23.

FIG. 25 illustrates a side view of the retractor delivery system of FIG. 23.

FIG. 26 illustrates a sectional view taken along line 26-26 of FIG. 25.

FIG. 27 illustrates a perspective view of a retractor delivery system formed in accordance with an additional embodiment of the present invention.

FIG. 28 illustrates a side view of the retractor delivery system of FIG. 27.

FIG. 29 illustrates a sectional view taken along line 29-29 of FIG. 28.

FIG. 30 illustrates a sectional view taken along line 30-30 of FIG. 28.

FIG. 31 provides a perspective view of a retractor blade fowled in accordance with an embodiment of the present invention.

FIG. 32 provides a sectional view of the retractor blade of FIG. 31 taken through a cross-section of a distal portion of the retractor blade.

### DETAILED DESCRIPTION OF THE INVENTION

Reference now will be made in detail to certain embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For example, features illustrated or described as part of one embodiment, may be used on or with another embodiment to yield a still further embodiment. Thus, it is intended that the present invention cover such modifications and variations. Other objects, features and aspects of the present invention are disclosed in or are apparent from the following detailed description. It is to be understood by one of ordinary skill in the art that the present discussion is a description of exemplary embodiments only, and is not intended as limiting the broader aspects of the present invention.

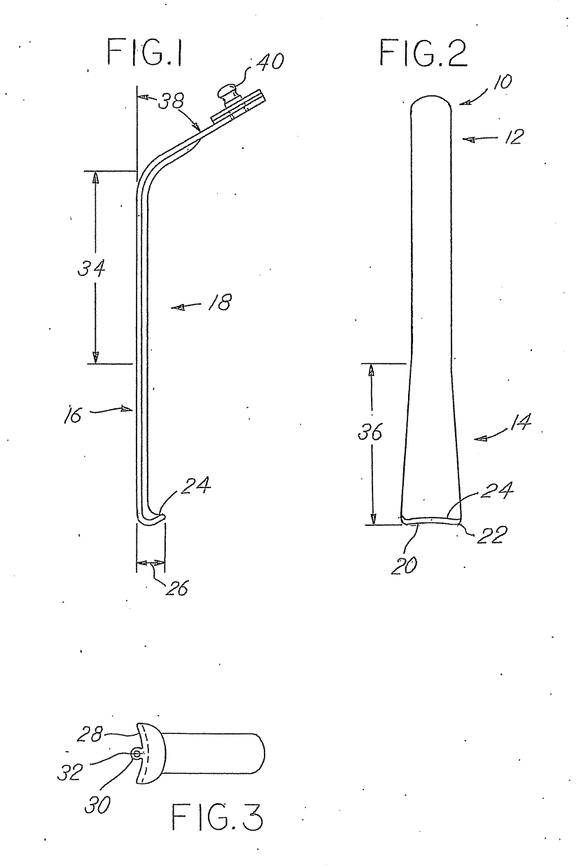

FIGS. 1 and 2 illustrate a side view and a rearward view, respectively, of a retractor blade 10 formed in accordance with an embodiment of the present invention that may be used, for example, in an anterior lateral procedure. The illustrated retractor blade 10 is adapted for use in lateral or anterior lateral spinal procedures. As with other components described below, the retractor blade 10 may be formed of, for example, stainless steel, to allow for sterilization and re-use. As best seen in FIG. 2, the retractor blade 10 includes a proximal portion 12 and a distal portion 14. The proximal portion 12 is oriented nearer the practitioner during a procedure, and the distal portion 14 is oriented inside the patient during the procedure. As best seen in FIG. 1, the retractor blade 10 also comprises a forward side 16 and a rearward side 18. The forward side 16 is oriented toward the inside of an incision when the retractor blade 10 is in place, and the rearward side 18 is generally oriented toward the perimeter of an incision when the retractor blade 10 is in place during a procedure.

As best seen in FIG. 2, proximate to the distal tip of the retractor blade 10, the retractor blade 10 includes a spinal engagement contour 20. The spinal engagement contour 20 is sized and adapted to allow improved conformance of the distal tip to a portion of the patient's spine. For example, the distal tip of the retractor blade 10 may include a spinal engagement contour 20 having a concave shape with a radius 22. For example, the radius 22 of the surface of the distal tip that contacts the spine when the retractor blade 10 is in place may be about 39.74 millimeters.

Proximate to the distal end of the rearward side 18 of the, retractor blade 10, the retractor blade 10 includes a lip 24. The lip 24 is sized and adapted to retain, or hold back, blood vessels, when the retractor blade 10 is in place during a procedure. For example, the lip 24 may extend in a curve from the forward side 16 to the rearward side 18 as illustrated in FIG. 1. The lip 24 defines a width 26 from the forward side 16 to the rearward edge of the lip 24. For example, the width 26 may be about 11 millimeters.

FIG. 3 illustrates an end view of the retractor blade 10 (looking upward from below the retractor blade as illustrated in FIG. 2.) As seen in FIG. 3, the retractor blade 10 includes a channel 30 including an opening 32. The channel 30 may run along a substantial length of the retractor blade 10. For example, the channel 30 in the illustrated embodiment may run along substantially all of the vertical portion of the retractor blade 10 as seen in FIG. 1. In alternative embodiments, the channel 30 of at least some of the retractor blades 10 used with a retractor blade system may be broken into multiple shorter lengths, such as a length proximate to a distal portion and a length proximate to a proximal portion of the retractor blade 10. The channel 30 is sized and adapted to accept mounting features of a retractor blade delivery system (discussed below). Additionally, the channel 30 may be adapted to accept a feature (such as a pin or screw) for mounting the retractor blade 10 in place to the spine during a procedure. As also seen in FIG. 3, a distal portion of the forward side 16 of the retractor blade 10 may be shaped to form a concave surface having a radius 28. The radius 28 may be shaped to conform to a surface of a delivery system and/or to improve access to a surgical site of interest. For example, the radius 28 may be about 25 millimeters.

Returning to FIGS. 1 and 2, the retractor blade includes a first length 34 and a second length 36. The first length 34 joins the second length 36, and the second length 36 is located distal to the first length 34. The sides of the retractor blade 10 are substantially parallel along the first length 34, and are flared wider along second length 36, allowing for greater surface area for holding back tissue closer to the surgical site, and greater maneuverability farther from the surgical site. The width across the first length 34 may be, for example, about 16 millimeters, and expand to about 24 millimeters at the distal end of the second length 36. The first and second lengths may be sized and adapted for different patient sizes and/or procedures. For example, the length of the second length 36 may be, for example, about 65 millimeters. The length of the first length 34 may be, for example, about 80 millimeters, about 100 millimeters, about 120 millimeters, or about 140 millimeters. Further, retractor blades may be provided as part of a kit including retractor blades having various first lengths. For example, a kit may include three retractor blades having a first length of about 80 millimeters, three retractor blades having a first length of about 100 millimeters, three retractor blades having a first length of about 120 millimeters, and three retractor blades having a first length of about 140 millimeters.

Toward the proximal end of the retractor blade 10, the retractor blade 10 includes a portion bending rearwardly at an angle 38. This portion is bent rearwardly to allow improved access to the site of interest as well as to facilitate securement of the retractor blade 10 to a retractor frame (not shown). For example, angle 38 may be about 60 degrees. Proximal to the proximal end of the retractor blade 10, the retractor blade includes a mounting post 40. The mounting post 40 is sized and adapted to be accepted by a clamp (not shown) for securing the retractor blade to a retractor frame after the retractor blade is in place during a procedure.

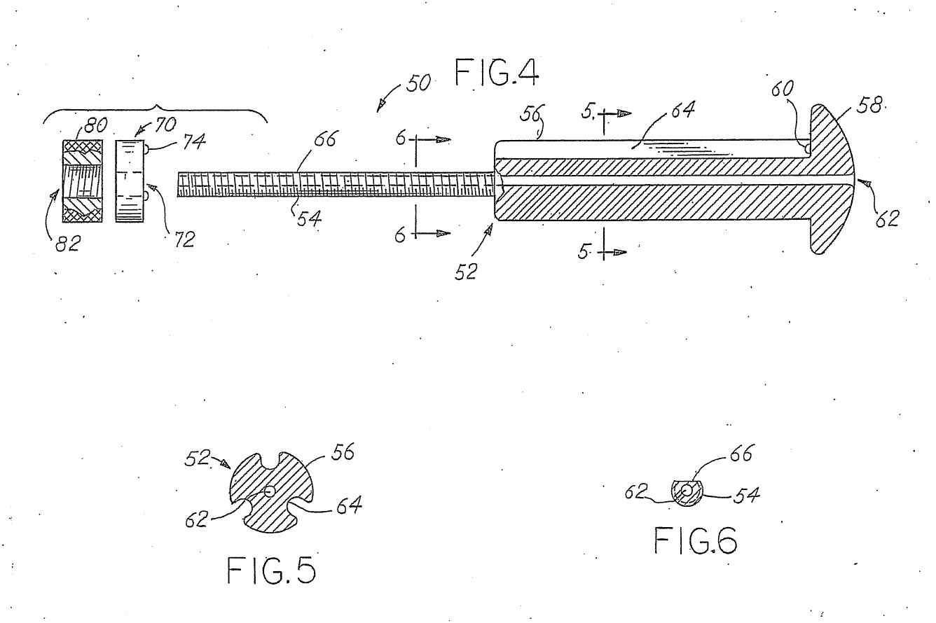

FIG. 4 illustrates a side view of a delivery system 50 for delivering retractor blades, such as retractor blades 10 described above, formed in accordance with an embodiment of the present invention. The embodiment illustrated in FIGS. 4-10 is for a system delivering three retractor blades. The delivery system 50 includes a shuttle 52, an anti-rotation collar 70, and a lock nut 80. The anti-rotation collar 70 and lock nut 80 provide one example of a securement mechanism/releasing mechanism for mounting retractors to a shuttle/releasing retractors from a shuttle. The shuttle 52 includes a threaded shaft 54, a retractor surface 56, a retractor groove 64, a head 58, a mounting feature 60, and a guidewire channel 62.

The shuttle 52 of the illustrated embodiment includes three retractor surfaces 56 and three retractor grooves 64 (to accommodate three retractor blades 10). The retractor surface 56 may be contoured to conform to the shape of the forward side 16 of the retractor blade 10 to allow the retractor blade 10 to be placed snugly against the shuttle 52 to ease entry of the shuttle 52 into an incision. The retractor grooves 64 are sized to accept the channel 30 of the retractor blade 10. A sectional view of the shuttle 52 taken along line 5-5 is illustrated in FIG. 5, showing the cross-section of the shuttle 52 and the shapes of the retractor surfaces 56 and retractor grooves 64 for the illustrated embodiment. Alternatively, a larger number of grooves and surfaces may be employed to allow use of a shuttle with differing numbers and/or sizes of retractors. For example, a shuttle 52 configured with 6 retractor surfaces and six retractor grooves may permit the attachment of three wider retractor blades, or six narrower retractor blades. The shuttle also includes a guidewire channel 62. The guidewire channel 62 is sized and adapted to accept a guidewire (not shown) for guiding the insertion of the shuttle 52 into an incision and toward the surgical site of interest.

The shuttle 52 includes a head 58 located proximate to the distal end of the shuttle 52. The head 58 is the leading portion of the shuttle 52 inserted into an incision, and is sized and shaped to protect the distal ends of the retractor blades 10 when they are in place on the shuttle 52, to protect both tissue of the patient and the retractor blades 10 from damage when the shuttle 52 is inserted. Alternate shapes of heads and retractor blades are possible. For example, when retractor blades without a lip, or with a smaller lip, are used, a head with a smaller cross-section may be used.

The shuttle 52 also includes three mounting features 60 (in the illustrated embodiment, one for each retractor blade 10). The mounting features 60 are sized and adapted to be accepted by the opening 32 of the channel 30 of the retractor blades 10, and cooperate with the opening 32 to maintain the retractor blades 10 in place on the shuttle 52 during placement into a patient during a procedure. For example, the mounting features 60 may be slight protuberances projecting from a proximally facing surface of the head 58.

The shuttle 52 also includes a threaded shaft 54. The threaded shaft 54 is sized and adapted to be accepted by the lock nut 80 in threaded engagement. The threaded shaft 54 also includes a flat surface 66 (see also FIG. 6 for a sectional view taken along line 6-6 illustrating the flat surface 66 in cross-section). The flat surface 66 is sized and adapted to cooperate with an opening of the anti-rotation collar 70 to help secure the retractor blades 10 in place on the shuttle 52.

FIG. 7 illustrates an end view of an anti-rotation collar 70 formed in accordance with an embodiment of the present invention. The anti-rotation collar 70 includes an opening 72 and mounting features 74. The opening 72 includes a flat surface 76 and is sized and adapted to be accepted by the threaded shaft 54 of the shuttle 52 with a clearance fit, and such that the flat surface 76 of the opening 72 cooperates with the flat surface 66 of the threaded shaft 54 to prevent rotation of the anti-rotation collar 70 with respect to the threaded shaft 54. The mounting features 74 are sized and adapted to be accepted by the opening 32 of the channel 30 of the retractor blades 10, and cooperate with the opening 32 to maintain the retractor blades 10 in place on the shuttle 52 during placement into a patient during a procedure. For example, the mounting features 72 may be slight protuberances projecting from a distally facing surface of the anti-rotation collar 70. In alternate embodiments, for example, the channel 30 may be reduced in size and/or removed, and the mounting features 74 (or another structure on the shuttle 52) may cooperate with holes located proximate to the top of the retractor blade (near the bend in the retractor blade).

Returning to FIG. 4, as also mentioned previously, the delivery system 50 includes a lock nut 80. The lock nut 80 includes a threaded opening 82 that accepts the threaded shaft 54 of the shuttle 52 in threaded engagement. The lock nut 80 acts to secure the anti-rotation collar 70 in place along a length of the threaded shaft 54 of the shuttle 52, thereby helping secure the retractor blades 10 in place when they are attached to the shuttle 52.

The placement and securement of retractor blades 10 to the shuttle 52 will now be described with reference to FIG. 8, which illustrates a sectional view of an embodiment of a delivery system with retractor blades attached formed in accordance with an embodiment of the present invention. FIG. 8 illustrates a retractor system 90 including retractor blades 10, a shuttle 52, an anti-rotation collar 70, and a lock nut 80.

To begin mounting a retractor blade 10 to the shuttle 52, the distal end of the retractor blade 10 is oriented toward the distal end of the shuttle 52, and the retractor blade 10 positioned such that the channel 30 of the retractor blade 10 is aligned inside a retractor groove 64 of the shuttle 52, and the forward side 16 of the retractor blade 10 is oriented toward the shuttle 52. The retractor blade 10 is then advanced toward the distal end of the shuttle 52, until the distal end of the retractor blade 10 abuts a proximally facing surface of the head 58, and the mounting feature 60 of the shuttle 52 is accepted by the opening 32 of the channel 30 of the retractor blade 10. This process is repeated for each retractor blade 10 (for example, three retractor blades total may be used).

With all retractor blades 10 in place, the anti-rotation collar 70, oriented so that the mounting features 74 face toward the distal end of the shuttle 52, is slid over the threaded shaft 54 of the shuttle 52, until it abuts surfaces of the retractor blades 10 (such as, for example, an end of the channel 30), and the mounting features 74 are accepted by the openings 32 of the channels 30 of the retractor blades 10. The lock nut 80 is then advanced in threaded engagement with the threaded shaft 54 into position against the anti-rotation collar 70 and urged forward to secure the anti-rotation collar in place. With the components of the delivery system 90 in place as described, the retractor blades are secured in place, oriented lengthwise along the shuttle 52, by the cooperation of the mounting features 60, 74 with the openings 32 of the channels 30.

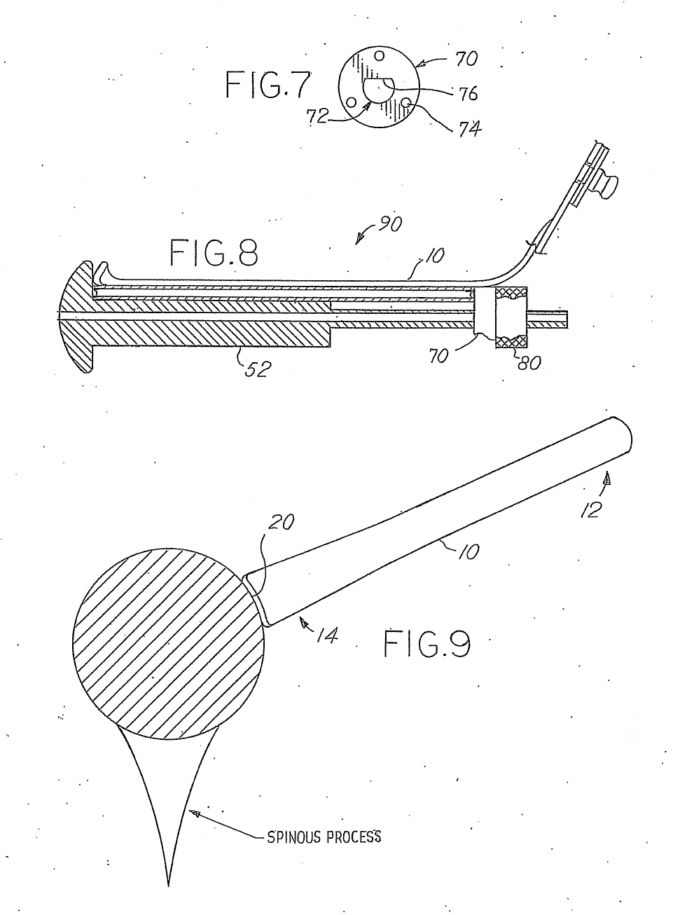

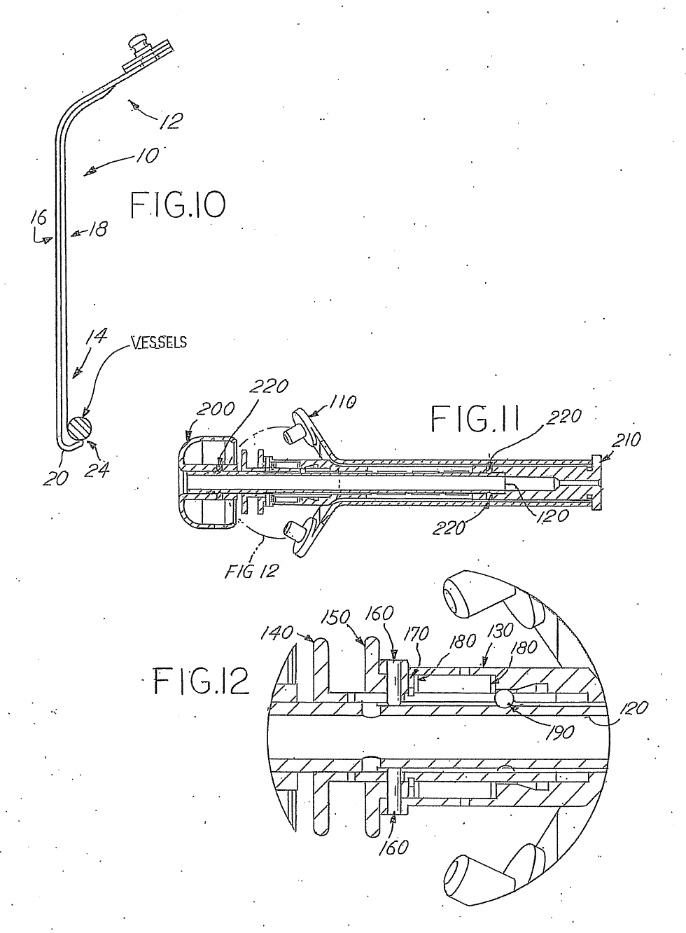

An example of the use of the retractor system 90 illustrated in FIG. 8 will now be described. An incision is made in a desired location to allow access to the surgical site of interest, in this example the spine of a patient. A guide wire is inserted into the incision and positioned as desired. The shuttle 52, with the retractor blades 10 secured in place as described above, in then inserted, distal end first, into the incision, with the guide wire passing through the guide wire channel 62. Thus, the guidewire is used to guide the shuttle toward the surgical site of interest. Once the shuttle 52 is advanced a desired distance into the patient, the lock nut 80 is loosened, allowing the anti-rotation collar 70 to be slid proximally far enough to allow the retractor blades 10 to be removed. A first retractor blade 10 is removed from the shuttle 52 and positioned as shown in FIG. 9, which illustrates an embodiment of a retractor blade that may be used, for example, in an anterior lateral procedure, formed in accordance with an embodiment of the present invention in place after being positioned during a spinal procedure. As shown in FIG. 9, the distal tip of the first retractor blade 10 is shaped to conform to the shape of the spine which the distal end of the retractor blade 10 abuts (FIG. 9 is a schematic used for simplified explanatory purposes and not intended to precisely illustrate the exact dimensions of a spine). As shown in FIG. 10, which illustrates a side view of a retractor blade formed in accordance with an embodiment of the present invention, the lip of the first retractor blade 10 may be used to hold back vessel(s) from the site of interest. Once the first retractor blade 10 is in position, it may be secured in place to the spine by, for example, a phi or a screw. The first retractor blade 10 may also be secured by a clamp accepting the mounting post 40 to an external retractor frame (not shown) that may be mounted, for example, to a hospital bed.

Next, additional retractor blades 10 are removed from the shuttle 52, positioned as desired, and secured to an external retractor frame. For example, two additional retractor blades 10 may be secured in place, generally with their forward surfaces facing each other, and facing perpendicularly to a direction extending from the forward surface of the retractor blade 10 that was first positioned. By using retractor blades that are removable from the shuttle, the blades may be individually and independently positioned, providing the ability to provide improved access to a site of interest, when compared to certain known dilators which do not provide the same amount of flexibility in sizing and/or shaping the access to the site of interest. Once all of the retractor blades 10 are positioned as desired, the shuttle 52 may be removed from the incision. The retractor blades 10 may then be removed once the procedure is completed.

Alternate embodiments of the blades and/or delivery system are also contemplated by the present invention. For example, FIGS. 11-16 illustrates a retractor system 100 formed in accordance with an embodiment of the present invention. The retractor system 100 includes retractor blades 110, a grooved shuttle shaft 120, a ball lifter 130, a shuttle ball retainer 140, a ball lifter collar 150, dowel pins 160, a retaining ring 170, spring 180, ball bearings 190, a shuttle handle 200, a shuttle insertion head 210, and set screws 220. The various components of the retractor system cooperate to secure and release the retractor blades 110.

FIG. 11 illustrates a side sectional view of the retractor system 100 adapted for delivery of different lengths of retractor blades, and FIG. 12 provides an enlarged view of a portion of the sectional view of FIG. 11. As illustrated, the ball bearings 190 are accepted by grooves in the grooved shaft 120, holes in the shuttle ball retainer 140, and a channel in the shuttle ball lifter 130. When the shuttle ball lifter 130 is positioned such that the ball bearings 90 are in a smaller diameter section of the channel of the shuttle ball lifter, the ball bearings 190 help hold the shuttle ball lifter 130 in position lengthwise along the grooved shaft 120. When the shuttle ball lifter 130 is positioned such that the ball bearings 190 are in a larger diameter section of the shuttle ball lifter 130, the shuttle ball lifter 130 may be moved lengthwise along the grooved shaft. The shuttle ball lifter collar 150 secures to the shuttle ball lifter 140 via dowel pins 160 and provides a surface for lifting the shuttle ball lifter 130 (thereby positioning the ball bearings 190 in the larger diameter portion and allowing movement) or lowering the shuttle ball lifter 130 (thereby positioning the ball bearings 190 in the smaller diameter portion and securing the ball lifter 130 in place). The shuttle ball retainer 140 extends along an inside portion of the shuttle ball lifter 130 to prevent unwanted migration of the ball bearings 190, in cooperation with washers 180, retaining ring 170, and grooves on the grooved shaft 120. The shuttle handle 200 and the shuttle insertion head 210 are secured to the grooved shaft with set screws 220.

FIG. 13 illustrates a side view of a shuttle ball retainer 140 formed in accordance with an embodiment of the present invention. The shuttle ball retainer includes holes 142 that accept the ball bearings 190, and a slot 144 that allows clearance for the dowel pins 160, allowing the shuttle ball lifter 130 and shuttle ball lifter collar 150 to move lengthwise along the grooved shaft 120 relative to the shuttle ball retainer 140 when actuated by a practitioner.

FIG. 14 illustrates a perspective view of the shuttle insertion head 210 formed in accordance with an embodiment of the present invention. The shuttle insertion head 210 is secured to and located distally of the grooved shaft, and includes retractor surfaces 212 and retractor grooves 214, which may be similar to those described above. The shuttle insertion head 210 also includes mounting features 216, sized and adapted to cooperate with openings on retractor blades (such as the openings of the channels discussed previously) to help secure retractor blades in place during insertion. In certain embodiments, the mounting features 216 are pins placed in pre-formed holes of the shuttle insertion head 210 during the manufacturing and/or assembly process. The shuttle insertion head 210 also includes a guidewire opening 218 to accept a guidewire for placement, similar to the above discussion.

FIG. 15 illustrates a side view of a grooved shaft 120 formed in accordance with an embodiment of the present invention. The grooved shaft 120 includes horizontal grooves 122 and vertical grooves 124. The horizontal grooves 122 cooperate with the holes 142 of the shuttle ball retainer 140 to maintain the ball bearings in place when the ball lifter 130 is in a secure position, and the vertical grooves 214 allow the ball bearings 190 to move lengthwise relative to the grooved shaft 210 when the ball lifter 130 is in a movable position. The grooved shaft 120 of the illustrated embodiment includes four horizontal grooves 122 to accommodate four different lengths of retractor blades 110.

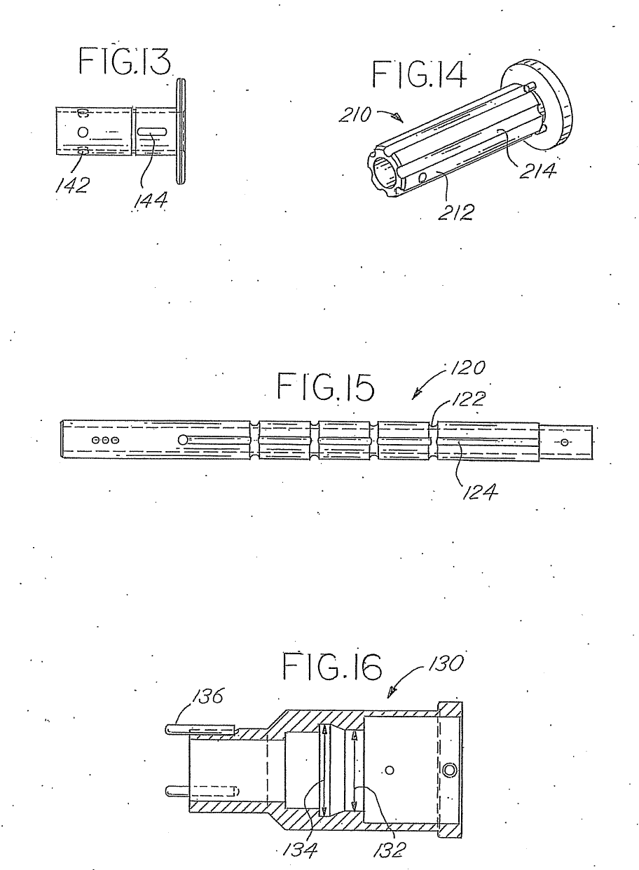

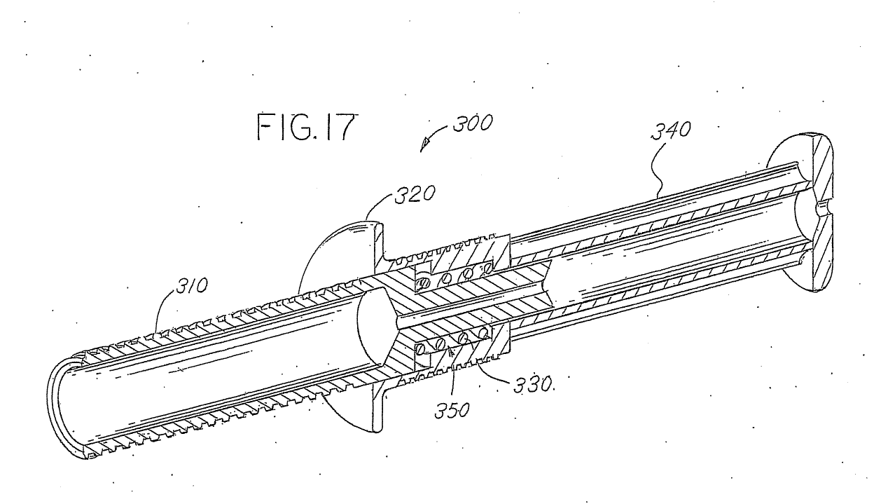

FIG. 16 illustrates a side sectional view of a shuttle ball lifter 130 formed in accordance with an embodiment of the present invention. As also discussed above, the shuttle ball lifter 130 includes a first diameter 132 and a second diameter 134. When the ball bearings 190 are positioned in the first diameter 132 (the smaller diameter), the ball bearings 190 are constrained and the shuttle ball lifter 130 and associated components are secured in place longitudinally along the grooved shaft 120. When the ball bearings 190 are positioned in the second diameter 134 (the larger diameter), the ball bearings 190 are not constrained, and the ball lifter 130 and associated components are in a movable position. The shuttle ball lifter 130 also includes slots into which mounting posts 136 may be fixed (for example, by welding), and the mounting posts 136 cooperate with an opening on the retractor blades 110 (for example, the openings of the channels discussed above) to secure the retractor blades 110 in place during insertion into an incision. The spring 180 acts to urge the shuttle ball lifter 130 and shuttle ball retainer 140 apart when the ball lifter collar 150 is not actuated by a practitioner, thereby maintaining the position of the ball bearings 190 in the first (smaller) diameter and securing the shuttle ball lifter 130 in a fixed position.

Use of the retractor system 100 is similar in certain respects to the above discussion with respect to retractor system 90. To mount the retractor blades 110, the ball lifter collar 150 is lifted, orienting the ball bearings 190 in the second diameter 134 of the ball lifter 130, allowing the ball lifter 130 (and its associated components) to be moved along the grooved shaft 120 proximally (i.e., away from the shuttle insertion head). The retractor blades 110 are then placed with their distal ends abutting a portion of the shuttle insertion head 210, and openings in the retractor blades 110 cooperating with mounting features 216. The ball lifter 130 is then advanced along the length of the grooved shaft 120 toward an appropriate horizontal groove 212 based on the length of the retractor blades 110, and returned to the secured position (i.e. with the ball bearings 190 positioned in the first diameter 132), with the mounting posts 136 of the ball lifter 130 cooperating with openings on the retractor blades 110 to hold the retractor blades 110 in place. To remove the retractor blades 110 after insertion, the ball lifter 130 is moved to the movable position by pulling on the ball lifter collar 150, and then moved distally to allow removal of the retractor blades 110.

FIG. 17 illustrates a sectional view of yet another retractor delivery system 300 formed in accordance with another embodiment of the present invention. The retractor delivery system 300 also allows for placement of different lengths of retractor blades (not shown). The retractor delivery system 300 includes a handle 310, a spring collar 320, a spring 330, and a blade holding aim 340 (that is positioned distally of the handle 310). The blade holding arm 340 is attachable to and removable from the handle 310, for example, by a threaded engagement, or, as another example, with set screws. The spring 330 is located in a cavity 350 defined between the spring collar 320 and handle 310, and urges the spring collar 320 toward the distal end of the blade holding arm 340. As discussed above, the blade holding arm may be sized and shaped to accept retractor blades. Also, a distally facing portion of the spring collar 320 and a proximally facing portion of a distal portion of the blade holding arm 340 may incorporate mounting features for securing retractor blades in place.

Use of the retractor delivery system 300 is similar in certain respects to the above discussion with respect to the other retractor systems. First, an appropriate length version of the blade holding arm 340 is selected, and secured to the handle 310 with the spring collar 320 interposed between the handle 310 and blade holding aim 340, and the spring 330 in place in the spring cavity 350. Then, the spring collar 320 is urged proximally, compressing the spring 330. With the spring collar 320 held back, the retractor blades are then positioned as desired along the blade holding arm 340, including aligning any cooperating mounting features. The spring collar 320 is then released, and the spring 330 urges the spring collar 320 distally toward the distal portion of the blade holding arm 340, securing the retractor blades in place. To release the retractor blades, the spring collar 320 is again urged proximally, allowing removal of the retractor blades.

FIG. 18 illustrates a perspective view of a retractor system 400 that may be used, for example, to provide a lateral approach to the spine, formed in accordance with another embodiment of the present invention, and FIG. 19 provides a side view. The retractor system 400 includes a shuttle 500 and retractor blades 600. Alternate embodiments of this retractor system are also contemplated. For example, retractor blades similar to those discussed previously may be modified and used with such a shuttle for either a lateral or anterior lateral procedure. Further, for example, a lateral procedure may be performed using the retractor blades without the shuttle.

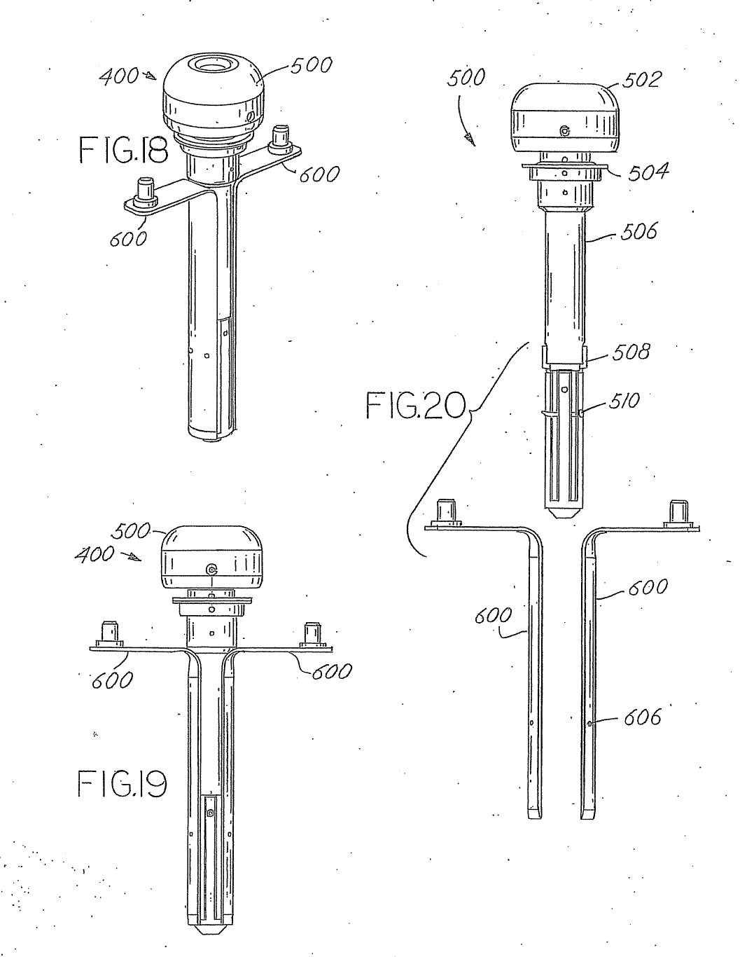

FIG. 20 illustrates a side view of the retractor system 400 with the retractor blades 600 removed from the shuttle 500. In the illustrated embodiment, the shuttle 500 includes a handle 502, a plunger 504, a movable body 506, first retention features 508, and second retention features 510. A spring (not shown) inside the movable body 506 urges the body 506 distally (in a downward direction as shown in FIG. 20). The plunger 504 provides a surface for a practioner to grasp and pull up on the movable body 506 against the spring, thereby bringing the movable body 506 and plunger 504 closer to the handle 502, and further from the distal end of the shuttle 500, which is not moved by the spring.

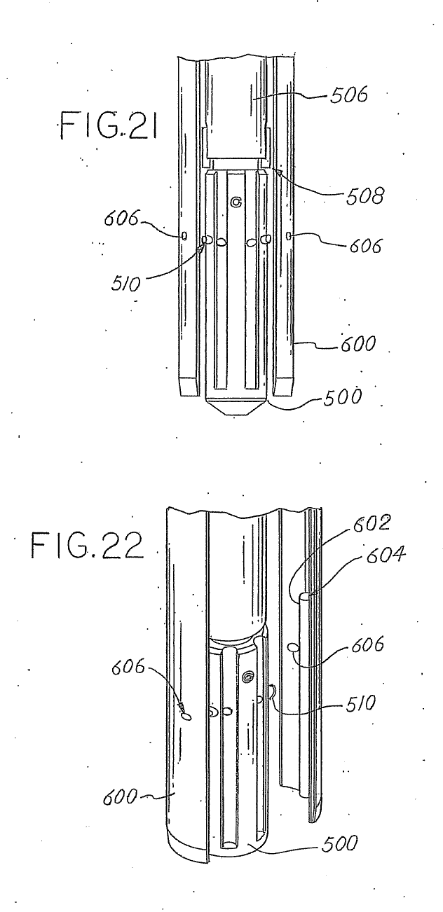

In the illustrated embodiment, each first retention feature 508 cooperates with a structure of the retractor blade 600 to help secure the retractor blade 600 to the shuttle 500, while a second retention feature 510 cooperates with another structure of the retractor blade 600 to resist the distal movement of the movable body 506 and retractor blade 600, thereby helping secure the retractor blade 600 in place on the shuttle 500. For example, as illustrated in FIG. 20 (and also shown in FIGS. 21-22), the first retention features 508 may be pins extending downwardly from a surface of the movable body 506 that are accepted by corresponding holes and/or tubes in the retractor blade 600, and the second retention features 510 may be pins extending outwardly from a portion of the shuttle 500 located distally from the movable body 506 that are accepted by corresponding holes in the retractor blade 600. For example, in the illustrated embodiment, the second retention features 510 extend at approximately 10 degrees above horizontal, and there are two second retention features 510 provided for each retractor blade 600. In alternate embodiments, for example, the first retention features 508 may be located at a different location along the shuttle 500, such as more proximally to engage with a feature located near the bend in the retractor blade 600.

In the embodiment illustrated in FIGS. 18-22, the retractor blades 600 do not have the same features proximate to their distal ends as the retractor blades previously described. The retractor blades 600 may be used, for example, in a procedure utilizing lateral access. As best seen in FIG. 22, a retractor blade 600 includes a tube 602 having an opening 604, and also includes a hole 606. The tube 602 and opening 604 are sized, positioned, and adapted to accept a first retention feature 508 of the shuttle 500. The hole 606 is sized and adapted to accept a second retention feature 510 of the shuttle 500. The tube 602 in the illustrated embodiment extends alone a center line of the retractor blade 600 from about its distal end to about halfway up the retractor blade 600. The tube 602 may also be used to receive a wire used to position the retractor blade 600. In the illustrated embodiment, there are two holes 606 for each retractor blade 600, equally spaced from and centered around the tube 602.

To use the shuttle 500 and retractor blades 600, first the retractor blades 600 are attached and secured to the shuttle 500. To accomplish this, the plunger 504 is urged toward the handle 502, thereby lifting the movable body 506 away from the distal end of the shuttle 500. With the plunger 504 lifted, the retractor blades 600 are positioned on the shuttle 500, with the second retention features 510 of the shuttle 500 accepted by the holes 606 of the retractor blades 600. Next, the plunger 504 is released, and the spring (not shown) urges the movable body 506 distally such that each of the first retention features 508 are accepted by an opening 604 of the tube 602 of each retractor blade 600. The spring force urging downward on the body 506 (and therefore as well on the retractor blade 600 via the cooperation of the shuttle 500 and the tube 602) urges the hole 606 of the retractor blade 600 against the second retention feature 510, and helps secure the retractor blades 600 to the shuttle 500. In alternate embodiments, a spring loaded mechanism may be located elsewhere on the assembly, or a non-spring loaded mechanism may be used to secure and release the retractor blades from the shuttle.

With as many retractor blades 600 as desired positioned on the shuttle 500 (two are shown in the illustrated embodiment), the shuttle 500 is introduced into an incision and positioned at or near a site of interest. The plunger 504 is then lifted such that the movable body 506 does not press downwardly on the retractor blades 600, and the second retention features 508 are clear of the tubes 604. The retractor blades 600 may then be removed from the shuttle 500 and placed as desired to provide appropriate access, and the shuttle 500 removed.

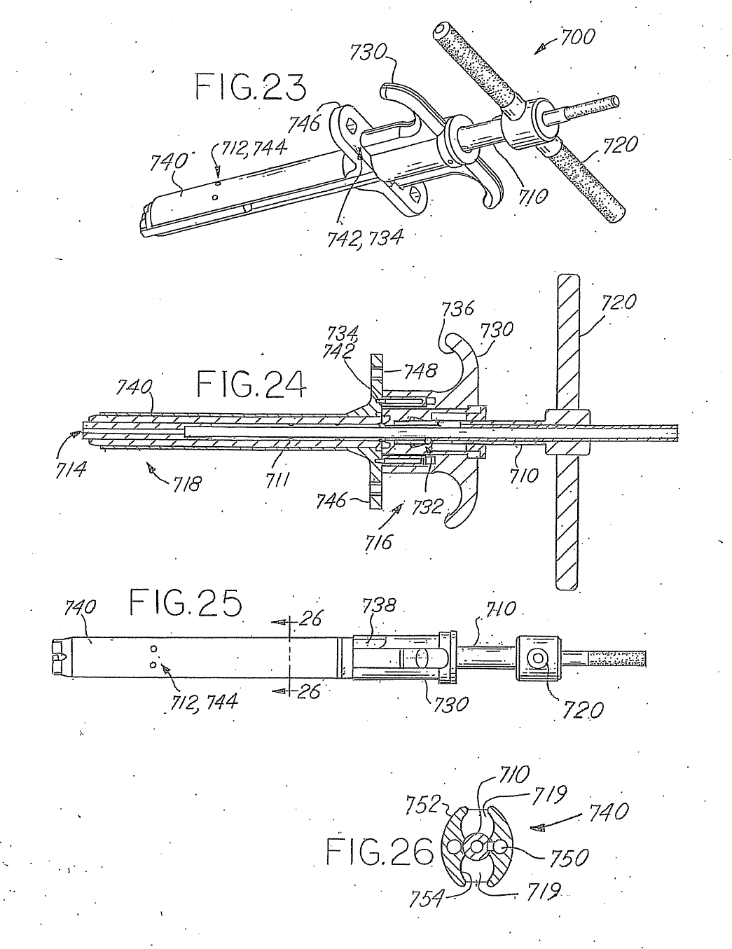

FIG. 23 illustrates a perspective view of another retractor delivery system 700 formed in accordance with another embodiment of the present invention. FIG. 24 provides a longitudinal sectional view of the retractor delivery system 700, FIG. 25 provides a side view of the retractor delivery system 700, and FIG. 26 provides a sectional view taken along line 26-26 of FIG. 25.

As shown in FIGS. 23-26, the retractor delivery system 700 includes a shaft 710, a handle 720, a release trigger 730, and retractor blades 740. In the illustrated embodiment, the retractor delivery system 700 includes two retractor blades 740. As best seen in FIG. 26, the retractor blades 740 have an arcuate outer surface, and when the retractor blades are positioned on the shaft 710, the retractor delivery system 700 has a generally oblong, oval, or “football” shaped cross-section. This oblong cross-section can ease insertion into an incision and improve use with an oblong incision. The handle 720 attaches to the shaft 710 and provides a convenient surface for grasping and manipulating the retractor delivery system 700 for use by a surgeon.

The shaft 710 may resemble previously discussed shafts in certain respects. The illustrated shaft 710 includes grooves 711, angled pins 712, a center hole 714, a proximal section 716, and a distal section 718. The proximal section 716 is oriented nearer to a practitioner when the retractor delivery system 700 is in place during a procedure, and the distal section 718 is oriented further inside the patient with the retractor delivery system 700 in place. The center hole 714 runs through the length of the shaft 710 and is sized to allow insertion of a guide wire, or K wire.

The proximal section 716 comprises a substantially cylindrical portion sized to accept the release trigger 730. The proximal section 716 also comprises grooves 711 that cooperate with ball bearings in the release trigger 730 to allow the release trigger 730 to be positioned at predetermined locations along the length of the proximal section 716, thereby allowing use of different lengths of retractor blades.

As best seen in FIG. 26, the distal section 718 of the shaft 710 comprises an oblong cross section comprising two generally cylindrical portions 719 on opposite sides, adjacent to and/or overlapping, a generally cylindrical portion extending from the proximal section 716. The overall shape of the cross section formed may be considered to be generally “Figure Eight” shaped. Each side of this cross section is shaped to correspond to and accept an inner surface of a retractor blade 740, so that a retractor blade 740 may be securely positioned lengthwise along the length of the shaft 710. The distal section 718 also comprises angled pins 712. The angled pins 712 are oriented at a slight angle above horizontal, and are sized, positioned, and configured to be accepted by holes in the retractor blades 740 to help secure the retractor blades 740 in place to the shaft 710.

The release trigger 730 is a mechanism that helps secure the retractor blades 740 to the shaft 710 and release the retractor blades 740 from the shaft 710. The release trigger 730 includes a central hole through which the shaft 710 passes. The release trigger 730 includes a ball lifter assembly 732, pins 734, a trigger 736, and an access hole 738 (see also FIG. 25). The ball lifter assembly 732 may comprise a mechanism including ball bearings similar, for example, to the ball lifter described above, that cooperates with a groove or grooves in the shaft 710 to position and secure the release trigger 730 in place along the length of the shaft 710. The trigger 736 is configured to provide a convenient surface for a practitioner's fingers to grasp the release trigger 730 and urge the ball lifter assembly 732 upward. Lifting upward on the trigger 736 (or, put another way, urging the trigger 736 proximally), releases the retractor blades 740 from the shaft 710.

The pins 734 extend generally perpendicularly away from a bottom surface of the release trigger 730, and are sized and positioned to cooperate with holes in the retractor blades 740 to help secure the retractor blades 740 in place. The access hole 738 is configured to allow passage of, for example, a probe for nerve stimulation.

The retractor blades 740 are used to hold back tissue to provide access to a surgical site of interest. In the illustrated embodiment, each retractor blade 740 includes an upper pin hole 742, lower pin holes 744, a handle 746, a mounting hole 748, an access hole 750, an outer surface 752, and an inner surface 754. The upper pin hole 742 is sized and configured to accept a pin 734 of the release trigger 730, while the lower pin holes 744 are angled, sized, and configured to accept angled pins 712 of the shaft 710.

The handle 746 is located proximally on the retractor blade 740, and is configured to provide a surface for manipulation and positioning of the retractor blade 740, and/or to provide a surface for securing the retractor blade. In the illustrated embodiment, the retractor blade 740 includes a mounting hole 748 passing through the handle 746. The mounting hole 748 is configured to cooperate with a clamp (not shown) to secure the retractor blade 740 in a desired position.

The retractor blade 740 also includes an access hole 750 passing generally lengthwise through a central portion of the retractor blade 740. The access hole 750 is positioned to align with an access hole 738 of the release trigger 730 to allow passage of, for example, a probe for nerve stimulation. The outer surface 752 of the retractor blade 740 is generally arcuate, and sized and configured to provide ease of entry into an incision and to hold back tissue to provide access to a surgical site of interest. The inner surface 754 of the retractor blade, as best seen in FIG. 26, is sized and adapted to correspond to the surface of the distal portion 718 of the shaft 720 so that the retractor blade 740 may be securely placed against the distal portion 718. For example, in the illustrated embodiment, the inner surface 754 defines two concave surfaces that accept convex surfaces of the distal portion 718, helping to provide aligning surfaces and prevent lateral movement of the retractor blade 740 relative to the shaft 718 while the retractor blade 740 is being secured in place.

Use of the retractor delivery system 700 may be generally similar in many respects to the above discussed examples. First, the two retractor blades 740 are positioned on the shaft 740, with the inner surfaces 754 placed against the corresponding surface of the distal portion 718 of the shaft 710, and positioned so that the lower pin holes 744 of the retractor blades 740 accept the angled pins 712 of the shaft 710. Next, the release trigger 730 is lowered into place, with the pins 734 of the release trigger 730 accepted by the upper pin holes 742 of the retractor blades 740. With the release trigger 730 secured in place, the various pins, holes, and surfaces described above cooperate to maintain the retractor blades 740 in place against the shaft 710. Next, an incision is made. The oblong cross-sectional shape of the distal portion of the retractor delivery system 700 with the retractor blades 740 secured provides improved insertion into an oblong incision. A stimulating probe may be inserted through an access hole or passageway through the retractor blades 740 to locate nerves to assist in the placement of the retractor delivery system 700. Further, after the desired positioning is determined, a K wire may be inserted through the shaft 710 and into a desired portion of the anatomy. An x-ray may be taken to verify the location of the retractor delivery system 700.

With the retractor delivery system 700, in place, the trigger 736 of the release trigger 730 may be actuated, allowing the retractor blades 740 to be slid off of the angled pins 712, and positioned as desired to provide the desired access to the site of interest. A retractor blade 740 may be secured in place inside the patient with, for example, a bone screw used in connection with the access hole 750 of the retractor blade 740, and may also be secured by securing the handle 746 of the retractor blade 740 to a clamp with the mounting hole 748. With the retractor blades 740 positioned as desired, the shaft 710 (along with the handle 720 and release trigger 740) may be removed.

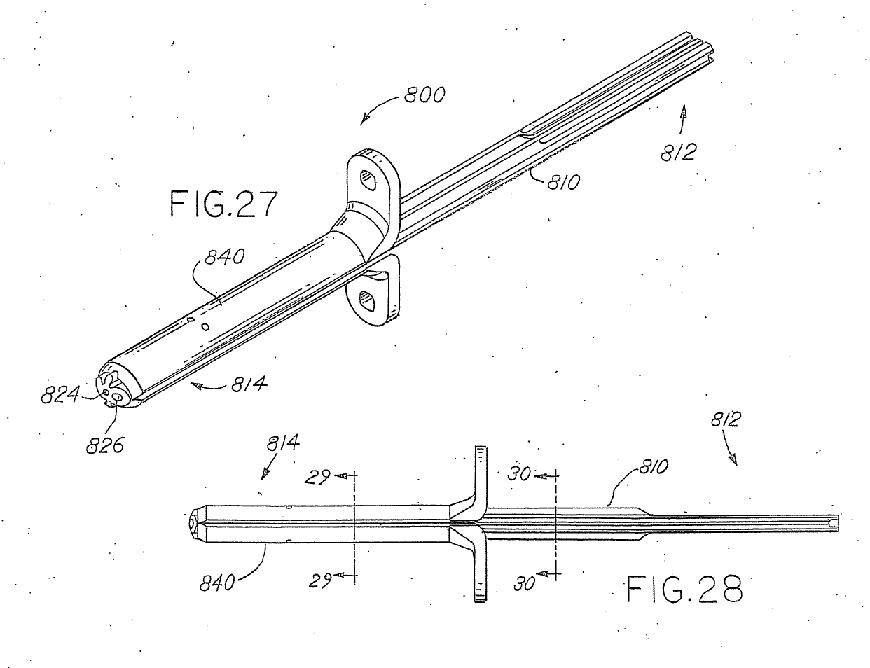

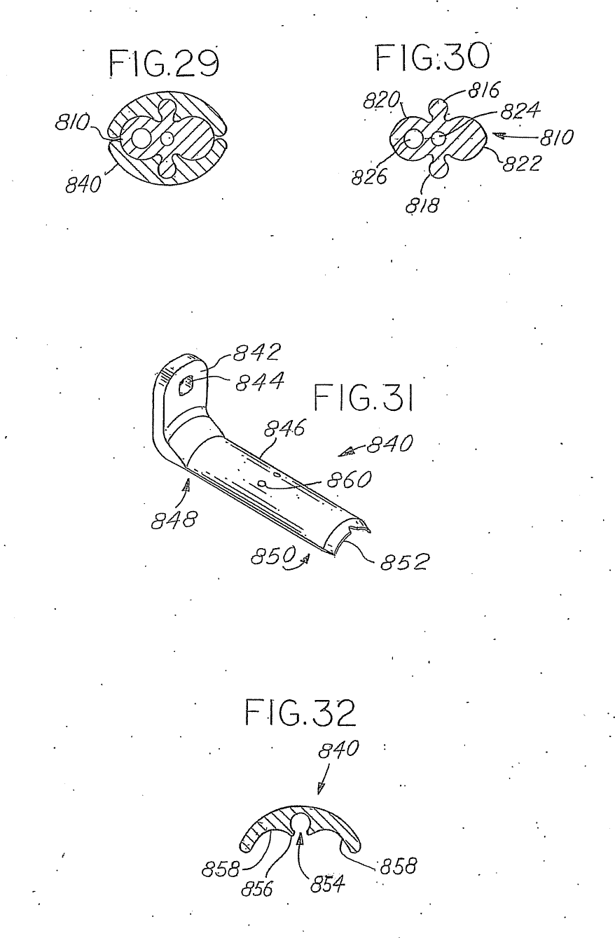

FIG. 27 provides a perspective view of a retractor delivery system 800 formed in accordance with an embodiment of the present invention, FIG. 28 provides a side view of the retractor delivery system 800, FIG. 29 provides a sectional view taken along line 29-29 of FIG. 28, and FIG. 30 provides a sectional view taken along line 30-30 of FIG. 28. As seen, for example, in FIG. 29, the retractor delivery system 800 has a generally oblong cross-sectional area. Thus, the retractor delivery system 800 is adapted to provide a reduced width for improved ease of insertion into an incision compared to a system with similarly sized retractors that is generally circular in cross section. The retractor delivery system 800 includes an oblong dissector 810 and retractor blades 840. As best seen in FIG. 29, the retractor blades 840 are slidingly engaged by aspects of the oblong dissector 810.

The oblong dissector 810, formed generally comprising a shaft along which retractor blades may be slid in the illustrated embodiment, includes a proximal end 812, a distal end 814, a first feature 816, a second feature 818, a third feature 820, a fourth feature 822, a center hole 824, and auxiliary hole 826. The distal end 814 is oriented into the patient when the retractor delivery system 800 is in use during a procedure, with the proximal end 812 nearer the practitioner. FIG. 30 provides a sectional view taken along line 30-30 of FIG. 28. As can be seen in FIG. 30, the first and second features 816, 818 are similarly sized, generally circular features symmetrically spaced about a central plane through the oblong dissector 810. For example, the first and second features, 816, 818 may include radiused surfaces having a radius of about 1.572 millimeters with the centers of the radiused surfaces offset about 5 millimeters on either side of a central plane. The overall shape of the third and fourth features, 820, 822 are also similarly sized to each other and symmetric about a central plane through the oblong dissector 810. The first, second, third, and fourth features 816, 818, 820, 822 are sized and configured to slidingly accept and guide the retractor blades 840 toward a surgical site of interest, as also discussed below.

As can be seen in FIG. 30, in the illustrated embodiment, a line passing through the centers of the first and second features 816, 818 is generally perpendicular to a line passing through the centers of the second and third features 820, 822. In the illustrated embodiment, the first, second, third, and fourth features 816, 818, 820, 822 are oriented such that they may be viewed as a type of “cloverleaf” with four leaves, two of which (corresponding to the third and fourth features 820, 822) are larger than the other. For example, in one embodiment, a width from the tip of the third feature 820 to the tip of the fourth feature 822 may be about 16.138 millimeters, while a width across the tips of the first and second features 816, 818 may be about 13.144 millimeters. As can be seen, for example, in FIGS. 27 and 28, aspects of the various features may be machined flat across portions of the proximal end 812 of the oblong dissector 810, for a reduced profile easing handling of the oblong dissector 810 and/or initial placement of the retractors 840 on the oblong dissector 810. Further, to ease insertion of the oblong dissector 810 into an incision, the tip of the distal end 814 may be radiused to provide a smooth and generally chamfered leading surface.

FIG. 31 provides a perspective view of a retractor blade 840, and Fig, 32 provides a sectional view of a retractor blade 840 taken through a cross-section of a distal portion of the retractor blade 840. The illustrated retractor blade 840 includes a handle 842 and a body 846. The handle 842 is configured to provide a convenient surface for grasping, manipulating, and/or securing the retractor blade 840 in place. The handle 842 includes a mounting hole 844 that is configured to allow securing of the handle 842 to a frame or other mounting system (not shown).

In the illustrated embodiment, the body 846 includes a proximal end 848, a distal end 850, a leading edge 852, a central channel 854, projections 856, curved surfaces 858, and angled pin holes 860. The angled pin holes 860 do not correspond directly to any aspect of the oblong dissector 810 for the illustrated embodiment. Instead, for the illustrated embodiment, they are included to allow use of a retractor blade 840 to be used with other retractor delivery systems as well. Thus, for example, a retractor blade similar to retractor blade 840 may be included as part of a kit or set including a number of blades as well as a number of delivery systems. As one example, the retractor blade 840 may be sized and configured to be mountable to the oblong dissector 810, as well as to be mountable to systems generally similar to certain embodiments of delivery systems discussed previously, such as retractor delivery system 700. The retractor blade 840 may also include additional pin holes and/or other features similar to those described above for retractor blades of previously discussed embodiments.

The handle 842 is joined to the body 846 at a portion of the proximal end 848 of the body 846. For example, the handle 842 may be welded to the body 846. In the illustrated embodiment, the body also includes a leading edge 852 located at the far end of the distal end 850 that is chamfered or beveled, and provides a smooth leading surface to be introduced into the incision to ease entry of the retractor blade 840 into an incision. Further, the tip of the leading edge 852 is slightly radiused to help avoid introduction of a sharp surface into tissue during insertion into an incision.

The central channel 854 runs generally along the length of the body 846, and is shaped to include a cross-section that is generally circularly shaped but open along a portion of the circle. For example, in the illustrated embodiment, the central channel 854 may include an arcuate portion having a radius of about 1.175 millimeters flanked by two projections 856. As may be seen in FIG. 29, the central channel 854 and projections 856 are sized and configured to slidingly accept either the first feature 816 or the second feature 818 of the oblong dissector 810, such that the retractor blade 840 may be slid longitudinally along a length of the oblong dissector 810, but substantial lateral movement relative to the oblong dissector 810 is prevented when the retractor blade 840 is engaged with the oblong dissector 810. The central channel 854 may also be sized and configured to accept a bone screw (not shown) to help fix the retractor blade 840 in place after it has been positioned to provide the desired access to a site of interest. The curved surfaces 858 are sized and configured to correspond generally to the third and fourth features 820, 822. For the illustrated embodiment, the curved surfaces 858 may include radiused surfaces having a radius of about 3.50 millimeters.

The use of the retractor delivery system 800 during a procedure will next be described. First, a practitioner makes an incision in the patient through which to insert the oblong dissector 810, distal end first. The oblong cross-sectional profile of the oblong dissector 810 allows, it to be oriented to more closely match the shape of the incision than implements having generally circularly shaped cross-section. A stimulating probe may be inserted into the auxiliary hole 826 to locate nerves during the insertion and/or placement of the oblong dissector 810. Once the desired positioning of the oblong dissector 810 is achieved, a K wire may be introduced through the center hole 824 and inserted into a disc of the spine to hold the dissector in place. Further, an x ray may be taken to verify the positioning of the oblong dissector 810.

With the oblong dissector 810 secured in a desired position, a first retractor blade 840 may be positioned on the oblong dissector 810 and slid, distal end first, into the incision down a side of the oblong dissector 810 to a desired position. Next, a second retractor blade 840 may be positioned and slid down the other side of the oblong dissector 810. The handles 842 of the retractor blades 840 may be secured to a frame or held by hand while still engaged with the oblong dissector 810, and then the oblong dissector 810 may be removed (in a sliding motion relative to the retractor blades 840). With the retractor blades 840 thus disengaged from the now removed oblong dissector 810, the retractor blades 840 may be positioned as desired, enlarging the access space provided to the practitioner, and secured in place. For example, a bone screw may be introduced into the central channel 854 of a retractor blade 840 to secure a distal portion of the retractor blade 840 to a portion of the patient's anatomy. Further, the handle 842 may be secured to a frame via a clamp cooperating with the mounting hole 844 of the retractor blade 840. With the two inserted retractor blades 840 positioned and secured, additional retractor blades, if desired, may also be introduced to restrain additional tissue from the site of interest.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present surgical port, which is set forth in the following claims. In addition, it should be understood that aspects of the various embodiments may be interchanged both in whole or in part. Therefore, the spirit and scope of the appended claims should not be limited to the description of the preferred versions contained therein.

What is claimed is:

1. A delivery system for introducing a plurality of retractor blades to a surgical site of interest, the delivery system including
a delivery device including a shaft having distal and proximal portions, the shaft including a plurality of engaging features extending along the distal portion of the shaft, the engaging features sized and configured to slidingly engage the retractor blades, wherein each retractor blade may be attached to the shaft independently by sliding onto one of the engaging features.

2. The delivery system of claim 1 wherein at least one of the engaging features comprises a generally circularly shaped portion configured to be accepted by a central channel of one of the retractor blades.

3. The delivery system of claim 2 wherein the shaft includes first and second curved surfaces on either side of the at least one of the engaging features, wherein the first and second curved surfaces are sized and configured to correspond to retractor curved surfaces located on either side of the central channel of the retractor blade.

4. The delivery system of claim 1 wherein the engaging features are symmetrically opposed about a central plane.

5. The delivery system of claim 1 wherein the proximal portion of the shaft defines a reduced profile with respect to the distal portion of the shaft.

6. The delivery system of claim 1 further comprising at least one lipped retractor blade, the lipped retractor blade comprising a distal portion, a rearward portion, and a forward portion opposed to the rearward portion, the rearward portion adapted to face toward a perimeter of an incision when the retractor blade is in place during a procedure, and the rearward portion including a lip configured to hold back vessels.

7. The delivery system of claim 6, wherein the distal portion of the lipped retractor blade comprises a distal tip shaped to abut a portion of a patient's spine.

8. The delivery system of claim 6 wherein the distal portion of the lipped retractor blade comprises a distal tip having an engagement contour having a concave shape with a radius.

9. The delivery system of claim 8 wherein the radius of the engagement contour is about 40 millimeters.

10. The delivery system of claim 6 wherein the lip extends in a curve from the forward side to the rearward side.

11. The delivery system of claim 1 further comprising at least one tapered retractor blade having a body, proximal end and distal end, the distal end comprising a leading edge having a reduced profile and providing a smooth leading surface to ease insertion of the tapered retractor blade into an incision.

12. A delivery system for introducing a plurality of retractor blades to a surgical site of interest, the delivery system including
a delivery device including a shaft having distal and proximal portions, the shaft including first, second, third, and fourth features extending along the distal portion of the shaft, the first and second features being symmetrically spaced about a first central plane and configured to slidingly engage a portion of one of the retractor blades, the third and fourth features being symmetrically spaced about a second central plane, wherein a line passing through the centers of the first and second features is generally perpendicular to a line passing through the centers of the third and fourth features such that the distal portion of the shaft defines a generally clover-leaf shaped cross-section; and
a plurality of retractor blades, each of the retractor blades comprising a central channel and curved surfaces located on either side of the central channel, the central channel sized and configured to slidingly engage one of the first and second features, the curved surfaces of the retractor blades being sized and configured to correspond generally to portions of the third and fourth features of the shaft, wherein each retractor blade may be attached to the shaft independently by sliding onto one of the first and second engaging features.

13. The delivery system of claim 12 wherein the first and second features are generally circularly shaped.

14. The delivery system of claim 12 wherein the central channel defines an arcuate portion flanked by two projections.

15. The delivery system of claim 12 wherein the third and fourth features are larger than the first and second features, wherein the distal portion of the shaft defines an oblong cross-section.

16. The delivery system of claim 12 wherein the proximal portion of the shaft defines a reduced profile with respect to the distal portion of the shaft.

17. A method for providing access to a surgical site of interest including
making an incision;
inserting, into the incision, a shaft including engaging features for slidingly engaging retractor blades;
inserting, into the incision, a first retractor blade by sliding the first retractor blade along one of the engaging features;
inserting, into the incision, a second retractor blade by sliding the second retractor blade along a different one of the engaging features than the engaging feature along which the first retractor blade was inserted;
removing the shaft while leaving the first and second retractor blades in the incision; and
positioning the first and second retractor blades.

18. The method of claim 17 further comprising securing a distal portion of at least one of the first and second retractor blades to a portion of a patient's anatomy.

19. The method of claim 17 wherein inserting the first retractor blade comprises positioning a central channel of the first retractor blade proximate to the one of the engaging features, wherein the one of the engaging features slidingly engages the first retractor blade, and inserting the second retractor blade comprises positioning a central channel of the second retractor blade proximate to the different one of the engaging features, wherein the different one of the engaging features slidingly engages the second retractor blade.

20. The method of claim 19 further comprising securing a distal portion of at least one of the first and second retractor blades to a portion of a patient's anatomy, wherein the securing comprises introducing a bone screw into the central channel of the at least one of the first and second retractor blades.
